(19) United States
(12) Patent Application Publication
(10) Pub. No.: US 2006/0058784 A1
(43) Pub. Date: Mar. 16, 2006

(54) Oxygen sensory system that minimizes outbreak of operating room fires

(76) Inventor: Tewodros Gedebou, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 10/940,330

(22) Filed: Sep. 14, 2004

(51) Int. Cl. A61B 18/14

(52) U.S. Cl. 606/45; 606/34

(57) ABSTRACT

Oxygen sensor systems that are effective in substantially reducing the risk of causing an operating room fire instigated by electrocautery surgical instruments and laser systems. The systems preferably include a conventional hand-held electrocautery instrument coupled with an oxygen sensor, the latter being operative to cutoff power to the electrocautery instrument or oxygen ventilation system once oxygen concentration levels are detected that exceed predetermined thresholds. In addition, the sensor would be coupled with an alarm system designed to warn the operating room staff of unsafe levels oxygen in the surgical environment. In further refinements of the invention, the oxygen sensor tip may be placed separate from the electrocautery instrument in the operative field, or even just outside the source of the oxygen to assure against excessive leaks. In still further refinements, the electrocautery device may be operative to shield the electrocautery tip once oxygen concentration levels are detected to exceed a predetermined threshold.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

Operating room fires and the hazards associated therewith are well known in the art. In this regard, hundreds of operating room fires arise annually during the performance of a variety of surgical procedures, resulting in dramatic tragedy to the affected patients and/or health care workers. Although all such cases are not reported due to liability issues, occasionally high profile cases have been publicized to this effect. In addition to the high fatality, several burn complications that result in lifelong suffering of the patients necessitates a solution for this problem.

Although a number of factors are known to contribute to the cause of operating room fires, such as flammable materials including alcohol prepping agents and gowns, the most significant etiologic factor for operating room fires is the combination of heat generated from electrically powered surgical equipment in the presence of oxygen. With respect to the former, it is well known that a variety of surgical equipment, and in particular electrocautery surgical instruments and lasers are known to emit substantial heat. Moreover, the tip of the electrocautery knife, due to the electrical current passing through, or the beam of the laser device is exceptionally prone to ignite a fire. A high concentration of oxygen and other flammable gases are also typically present, particularly during surgical procedures involving the head and neck insofar as oxygen tends to build beneath the surgical drapes or in the oropharyngeal cavity, which thus are operative to create an oxygen-enriched combustible atmosphere. Under such conditions, materials that are not considered flammable in normal circumstances can easily ignite with the resultant fire burning more violently and/or at higher temperatures. Although there are many methods of delivery of oxygen to the patient, there is currently no means of monitoring any oxygen leaking throughout the procedure.

Despite the well-known hazards associated with performing surgery under such conditions, however, there has not heretofore been any effective type of system or method that is operative to minimize the potential outbreak of operating room fires or even reduce the impact of the fire when it erupts. In this regard, the best safety practices currently in use merely involve taking precautionary measures and typically consist of nothing more than making efforts to minimize the build up of oxygen and nitrous oxide, activating electrosurgical and electrocautery units at lower power settings, and/or avoiding surgical draping. Additional precautions include turning equipment off when not in use or otherwise placing electrosurgical instruments in a safe location, such as a safety holster, when not in active use. Likewise recommended is the practice of allowing a certain amount of time, such a minute or more, to discontinue oxygen administration to the patient prior to the use of the electrosurgical instruments, lasers and the like. As such constant vigilance and communication between the anesthesiologist in charge of oxygen delivery and the surgeon in charge of the electrosurgical instruments is necessary.

Notwithstanding such safeguards, even the best practices are not effective to substantially reduce the risk of operating room fires. In this regard, there is simply no system or method currently available that enables high-risk surgical equipment, and in particular electrosurgical instruments such as electrocautery pin knives, lasers and the like, to be effectively utilized in oxygen-enriched environments while at the same time effectively eliminate the potential for such elements to create a fire hazard. There is likewise substantially lacking in the art any type of system and method for reducing the risk of operating room fires that can be readily integrated as part of an existing electrosurgical device, and in particular an electrocautery cutting apparatus that can be utilized per conventional electrocautery instruments and be utilized per conventional electrosurgical instruments for use in performing a wide variety of surgical procedures. There is likewise no system of determining if unsafe levels of oxygen are accumulating in the operative field. There is likewise a need for such a system and method that is of simple construction, exceptionally low cost, very safe to utilize and can be constructed utilizing well-known, commercially available materials.

### BRIEF SUMMARY OF THE INVENTION

The present invention specifically addresses and alleviates the above-identified deficiencies in the art. In this regard, the present invention is directed to an oxygen sensor system that substantially reduces, if not eliminates, the potential for an outbreak of an operating room fire by warning of unsafe levels of oxygen and disabling either the ignition source or the oxygen delivery. In a first embodiment, the system comprises a conventional electrocautery or laser instrument operatively coupled with an oxygen sensor, the latter being operative to determine the concentration of oxygen in the environment surrounding the distal-most operative end of the instrument. Such oxygen sensor is operative to cut the power delivered to the electrocautery instrument to the extent the concentration of oxygen surrounding the distal-most operative end of the electrocautery instrument exceeds predetermined thresholds. In a further refinement of such embodiment, the oxygen sensor would be operatively coupled to the power level switch utilized to set the intensity of the electrocautery instrument and further be operative to serve as a cutoff switch to the extent the surrounding oxygen concentration, coupled with a particular power setting utilized by the electrocautery instrument, meets or exceeds predetermined levels. In further refinements, an alarm system would be set to sound off when oxygen levels are reaching unsafe levels so as to warn the anesthesiologist to reduce the oxygen delivery and the surgeon to halt use of the electrosurgical apparatus. According to such embodiment, such oxygen sensor will be operative to terminate operation of the electrocautery instrument to the extent either oxygen concentration levels are too high and/or operation of the electrocautery instrument is operating at a power intensity that is likely to ignite a flammable material. In a similar fashion, the sensor could be coupled with an automatic shutoff mechanism to the release valve of the oxygen delivery system. In a further embodiments, the oxygen sensor may be placed separately near the operative site on the patient's body, under the surgical drapes or just external to the breathing tubes.

In a further refinement to enhance the ability of the electrocautery instrument to resist igniting an operating room fire, the electrocautery instrument of the present invention may be provided with a mechanical mechanism that prevents the heat or spark generated from the electrocautery tip from coming into contact with an oxygen-enriched environment once the concentration of oxygen in the area surrounding the distal-most end of the electrocautery tip reaches a predetermined concentration. In the first of two preferred embodiments, the distal-most tip of the electrocautery instrument will be provided with a shell mechanism that is axially positioned about the distal-most end of the electrosurgical instrument. The shell is operatively transitional between a first retracted configuration wherein the electrocautery blade is allowed to extend therefrom and be utilized during the surgical procedure. Such shell will have an aperture concentrically disposed thereon that is operative to enable the electrocautery blade to extend therefrom during the performance of a surgical procedure. To provide protection against fire ignition, the shell can transition to assume a second extended configuration, wherein the shell extends over and contain the electrocautery blade therein so that the tip is not exposed to the outside environment. Such shell is operatively coupled to the oxygen sensor and, once the oxygen sensor determines that the oxygen concentration about the distal-most end of the electrocautery instrument reaches or exceeds a given threshold, will cause the shell to extend about it and thus prevent the tip from coming into contact with any type of ignitable substance. Such protective covering, as defined by the extended covering about the electrocautery tip, provides an additional level of protection.

In the second of such embodiments, the electrocautery tip is operatively coupled to a solenoid disposed within the electrocautery instrument housing. Upon application of a current to the solenoid, the electrocautery tip is caused to transition from a first operative configuration, whereby the electrocautery tip is allowed to extend from the electrocautery housing for use in performing a surgical procedure, and a second retracted configuration wherein the electrocautery is withdrawn within the housing and thus safely contained therein. In the latter configuration, the housing surrounding the distal-most opening is operative to protect the tip and minimize the potential for the electrocautery device to ignite an operating room fire.

In yet further refinement of the invention, there may be provided manual switching means to activate either the protective shell or solenoid-type embodiments so that the electrocautery tip is protected. This may also involve a retractable clear protective housing element that allows visualization of the tip while protecting the spark formation. As such this guard would protect the patient from accidental activation related burns to the body.

It is therefore an object of the present invention to provide an oxygen sensory device that is operative to substantially reduce, if not eliminate, the potential to cause an operating room fire as well as reduce the impact of injury.

Another object of the present invention is to provide an electrocautery surgical device that is of simple design, easy to utilize, utilizes safe and commercially available materials, and will not interfere with a surgeon's ability to perform a surgical procedure and the anesthesiologist's ability to provide adequate oxygenation to the patient.

Another object of the present invention is to provide an electrocautery surgical device that can be utilized in a safe and effective manner that further reduces the chance of igniting an operating room fire but that further does not harm the patient in any way.

Still further objects of the present invention is to provide an electrocautery surgical device, as well as methods for using an electrocautery surgical instrument in a manner that substantially reduces, if not eliminates, the possibility of igniting an operating room fire, that can be readily implemented utilizing safe, low cost technology, and can be readily implemented utilizing well-known and commercially-available technology.

### BRIEF DESCRIPTION OF THE DRAWINGS

These as well as other features of the present invention will become more apparent upon reference to the drawings.

FIG. 1 is a perspective view, a portion of which shown in cross-section, of a system utilizing an oxygen sensor electrocautery system that is operative to substantially minimize the potential for the outbreak of an operating room fire while such system is utilized to undertake a surgical procedure.

FIG. 2 is a perspective view of the various other places within the operative field the oxygen sensor may be placed.

FIG. 3 is a cross-sectional view of an electrocautery instrument depicting the distal-most end therein being operatively transitional between a first retracted configuration and a second protective configuration.

FIG. 4 is a cross-sectional view of an electrocautery surgical instrument constructed in accordance with a second preferred embodiment of the present invention, electrocautery instrument being shown in a first operative configuration.

FIG. 5 is a cross-sectional view of the electrocautery instrument of FIG. 4 wherein said instrument is shown assuming a second protective configuration operative to minimize the risk of an outbreak of an operating room fire.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

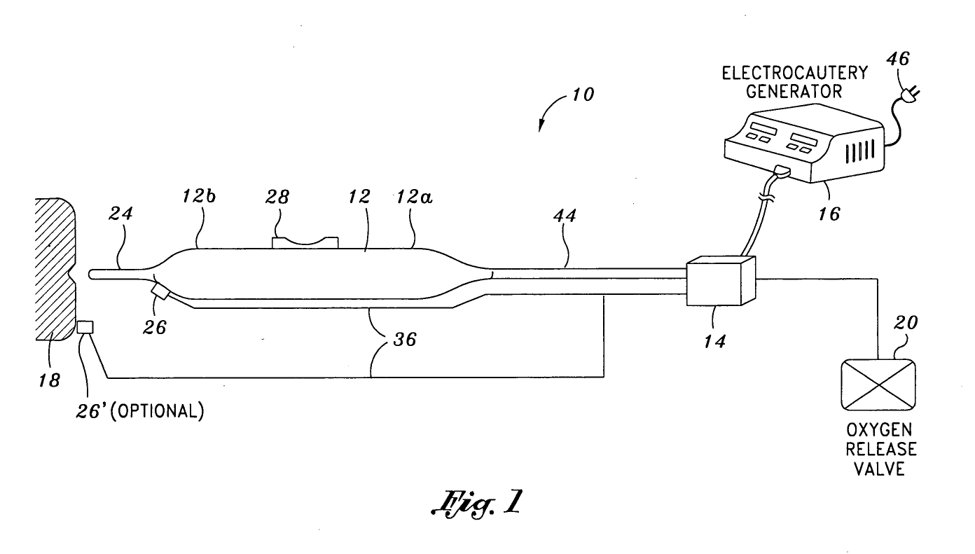

Referring now to the figures, and initially to FIG. 1, there is shown an electrosurgical system 10 for performing electrocautery surgical procedures that substantially reduces, if not eliminates, the possibility for such system 10 to ignite or otherwise cause and operating room fire. As shown, the system 10 comprises three essential components, namely, a hand-held electrocautery cautery instrument 12, a control unit 14, and an electrocautery generator 16 and an oxygen sensor 26.

With respect to the electrocautery instrument 12, as per conventional electrocautery instruments, the same is preferably formed as an elongate pen-knife having a proximal end 12a, and a distal end 12b, which is oriented toward the surgical site to which the electrocautery instrument is utilized, such as to coagulate bleeding vessels or cut through tissue 18. Formed on the distal-most end of the instrument 12a is a cautery tip 24 that is operative to be extended from the distal-most end instrument 12 to thus enable the same to cauterize a given site of tissue. To achieve that end, the cautery instrument 12 is coupled to control unit 14 via cord 44 and ultimately to electrocautery generator 16, the latter being coupled to an external power source provided at 46. As per conventional electrocautery devices, electrocautery device 12 is provided with a switch 28 that is operable by hand or foot (not shown), to thus selectively actuate the cautery tip 24 thereof.

Unlike conventional electrocautery devices, however, the electrocautery device 12 is further provided with an oxygen sensor 26 that is operative to measure the concentration of oxygen present in the vicinity surrounding the distal-most end 12b of the electrocautery instrument 12. Such oxygen sensors, which are well-known in the art and commercially available, are operative to determine the concentration of oxygen and generate and send a signal corresponding thereto. With respect to the latter, such signal is transmitted via link 36 to control unit 14. To the extent the concentration of oxygen detected by oxygen sensor 26 meets or exceeds a predetermined threshold, a signal will be transmitted via link 36 to control unit 14 that is operative to independently cause the control unit 14 to switch off all or a portion of power provided by the electrocautery generator 16, via power link 44, to the electrocautery instrument 12 to thus serve as an emergency shut off switch to the extent oxygen concentration levels meet or exceed a predetermined threshold. In this respect, it is contemplated that the oxygen sensor 26 will thus act as a circuit breaker to thus prevent the electrocautery tip 24 to ignite an oxygen-enriched environment once the concentration of oxygen reaches a predetermined threshold.

A further refinement of the system would include a link between the control unit 14 and an oxygen release valve 20. Thus, as the oxygen levels reach unsafe levels, as determined by the oxygen sensors, the control unit 14 would shut the valve off and thereby prevent the delivery of oxygen to the operative field.

In addition to or separate from oxygen sensor 26 placed upon the distal end of the electrocautery instrument 12, there may be utilized oxygen sensor 26′ that is placed upon the site 18 at which the electrocautery surgical procedure is performed. Along these lines, and as discussed more fully below, it is contemplated that such oxygen sensors 26, 26′ may be selectively positioned about the operative field to thus comprehensively asses the oxygen concentration levels so that the oxygen concentration levels can be comprehensively monitored throughout the surgical procedure.

In addition, an alarm system could be incorporated into the control unit 14 such that it would sound off a warning when the oxygen concentration reaches predetermined unsafe levels of oxygen or if the rate of increase in ambient oxygen levels is too high. In such manner, it is anticipated that the anesthesiologist adjust the oxygen delivery accordingly and the surgeon refrain from use of the electrosurgical or laser device.

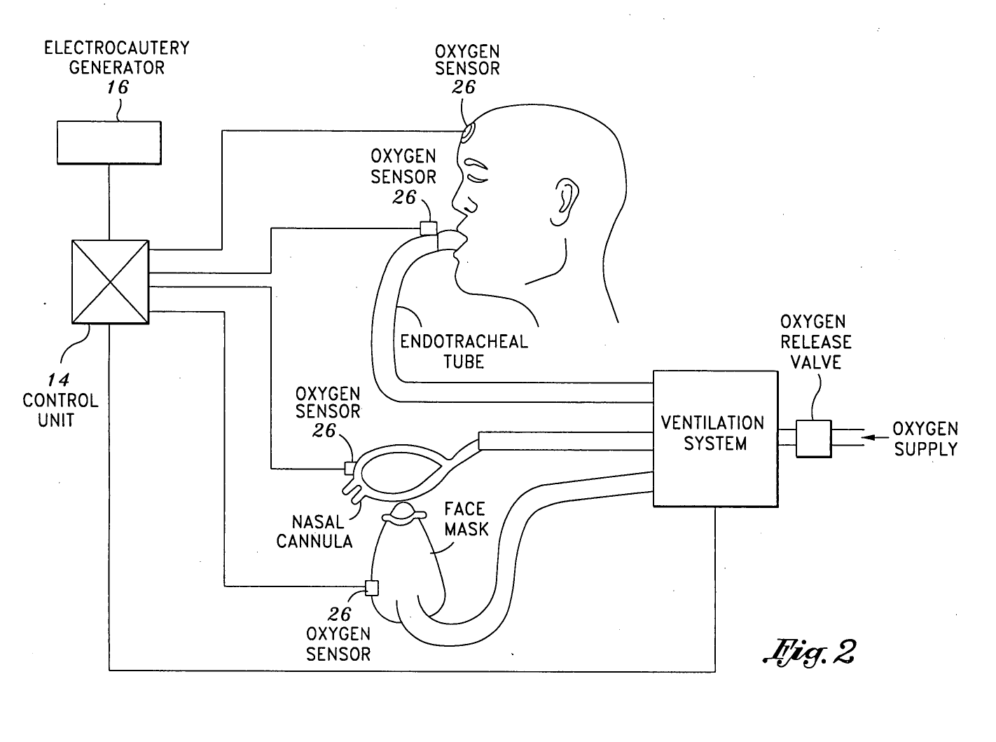

Referring now to FIG. 2, there is shown a multiplicity of locations where the oxygen sensor 26 may be placed within the operative field, which included the area about the patient, patient gowns, operating table, and the like, such that when oxygen levels at a particular site reach or exceed a predetermined threshold, would be operative to send a signal to the control unit 14, the latter being in electrical communication with electrocautery generator 16, to thus operate in the aforementioned manner whereby power supplied to an electrocautery instrument is cut off or substantially reduced thus eliminating the outbreak of fire. As illustrated, the oxygen sensor 26 may be placed upon a patient, such as by the patient's forehead 26, at the distal-most end of an endotracheal tube entering the patient's airway, upon a nasal canella, in particular at or near the juncture at where such nasal canella enters the nostrils of the patient, and upon an oxygen face mask. With respect to the latter three placement areas, namely, the endotracheal tube, nasal canella, and face mask, the same are closely linked to the point at which oxygen is delivered to the patient. Accordingly, by providing oxygen sensors 26 at such locations will advantageously provide the ability to detect oxygen levels where they are most likely to be their highest and/or where the greatest amount of oxygen leak can occur, thus making such distinct locations more vulnerable to the outbreak of an operating room fire. Along these lines, it is contemplated that the oxygen sensor 26 can be placed in an of a variety of areas on or about a patient and further, that multiple oxygen sensors 26, such as both sensors 26 and 28 of FIG. 1, may be utilized to comprehensively assess the oxygen levels about a patient while such electrocautery instrumentation is utilized.

In yet a further safety enhancement afforded by the systems of the present invention, the electrocautery device 12 may further include a mechanical safeguard mechanism that is operative to form a physical barrier about the distal cutting tip 24 of cutting element 22 to further reduce the risk of igniting an operating room fire. Referring now to FIG. 3, there is shown the first of two embodiments that are operative to provide a protective covering about cutting tip 24 once oxygen concentration levels are detected by oxygen sensor 26 that meet or exceed a predetermined threshold. According to such embodiment, the distal-most end 12b of electrocautery device 12 includes a shell or shield member 34 that is attached thereabout. Such shield member 34 will preferably be formed to have a bowl-like or frusto-conical shape with an aperture axially formed concentrically therein to thus define an aperture through which cutting element 24 may extend. The shield 34 is operatively transitional between a first retracted configuration, whereby the shield 36 is maintained in close proximity to the distal end 12b of the electrocautery device 12. To the extent oxygen concentration levels are detected by oxygen sensor 26 (as shown disposed within the device) that meet or exceed certain predetermined thresholds, a signal will be transmitted to control unit 14 via link 36 which in turn will cause a return signal to be generated from link 38 to switch 32, the latter being operative to cause the shield 34 to operatively transition from its retracted configuration to a second extended configuration, shown in phantom in FIG. 2, to thus extend about and form a covering around cutting tip 24.

In this regard, switch 32, which may be either formed externally from the electrocautery instrument 12, (or internally, as depicted), will cause shield 34 to advance in the direction indicated by the letter “A” such that a void or compartment, as shown as 52 in FIG. 3, around the cutting tip 24 to thus act as a physical barrier to prevent the cutting tip from coming into contact with a flammable material.

As will be appreciated by those skilled in the art, in order to accommodate such structure, it is contemplated that the various electrical connections, such as connection between switch 32 and link 38 and links 30 and 36 will be internally disposed within the internal compartment 20 defined by the electrocautery device 12 housing. It is likewise contemplated that the means by which protective shield 34 will transition from its first retracted configuration to its second protective, operative configuration may be accomplished by a variety of techniques known in the art, such as via spring activation or electromechanical means, such as through the activation of a solenoid or the like. To that end, and in order to ensure that cautery element remains axially disposed within the electrocautery device 12, it is contemplated that a support structure, such as 50 will be provided that will be operative to concentrically orient element 22 such that cautery tip 24 is shielded within 34.

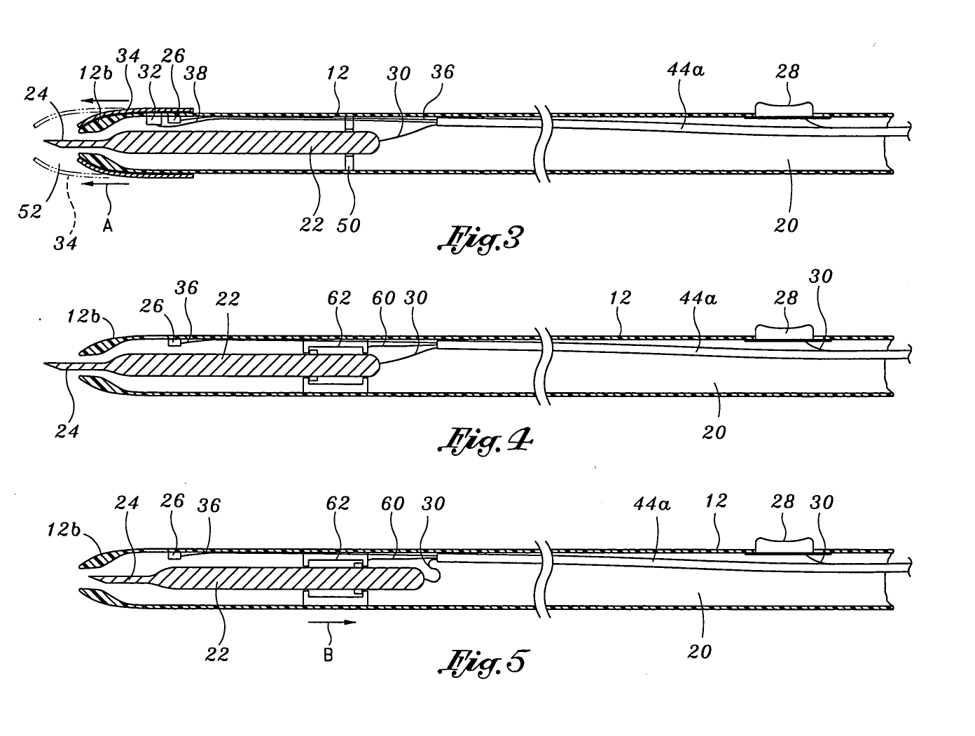

Referring now to FIGS. 4 and 5, there is shown an alternative embodiment whereby cautery tip 24 of cautery element 22 is operative to be protectively concealed within the electrocautery device 12 in the event the oxygen sensor 26 detects oxygen concentration levels that meet or exceed a predetermined threshold. Referring initially to FIG. 3, there is shown the operative configuration of the electrocautery instrument 12 with the cautery element 24 extending through the distal end 12b thereof.

To the extent the oxygen sensor 26 detects elevated oxygen concentration levels, a signal corresponding thereto will be transmitted via link 36 to control unit 14 (not shown) which will in turn activate a current to pass through electrical links 30, 60 to thus cause the solenoid 62 to cause the cautery element 22 to retract in the direction indicated by the letter “B”, within the interior 20 of the electrocautery device 12, as shown in FIG. 4. Similar to the embodiment depicted in FIGS. 1 and 2, the retraction of cautery element 22 within the interior 20 of electrocautery device 12.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention. In this regard, it is contemplated that the systems of the present invention need not include all of the specific safety features specified herein, namely, the use of an automatic power shut off, an automatic oxygen release valve shut off, an alarm system and/or shielding of the electrocautery tip, but may use only one such safety mechanism, or combination of any of the mechanisms.

What is claimed is:

1. An oxygen sensor system for minimizing the outbreak of an operating room fire comprising:
a) an electrocautery device having a proximal end and a distal end for electrically cauterizing tissue;
b) a control unit operatively coupled to said electrocautery device, the control unit being coupled to a power source to controllably apply power to said electrocautery device;
c) an oxygen sensor operatively coupled to said control unit, the sensor being operative to determine the oxygen concentration at the distal end of the electrocautery device; and
d) wherein the oxygen sensor is operative to transmit a signal to said control unit operative to shut off power supplied from said control unit to said electrocautery device when said oxygen sensor detects a concentration of oxygen above a threshold level.

2. The oxygen sensor system of claim 1 further comprising:
a) a source of oxygen for ventilating a patient upon which a surgical procedure is performed using said electrocautery device, the oxygen source having a release valve operatively coupled thereto to effect an emergency shut off of said source of oxygen; and
b) wherein said release valve is operatively coupled to said oxygen sensor, said release valve being operative to shut off oxygen supply from said source of oxygen when said oxygen sensor detects a concentration of oxygen above a threshold level.

3. The system of claim 1 wherein said oxygen sensor is operative to determine the oxygen concentration at the surgical site at which the electrocautery device is deployed.

4. The system of claim 2 wherein said system further including multiple oxygen sensors to determine the oxygen concentration in the operative field.

5. The system of claim 2 wherein said oxygen sensor determines the oxygen concentration at the point at which said source of oxygen is administered to said patient.

6. The system of claim I wherein said oxygen sensor is operative to activate an alarm when said oxygen sensor detects a concentration of oxygen above a threshold level.

7. The system of claim 1 wherein said system further includes:
a) a shielding mechanism for forming a physical barrier about said distal end of said electrocautery device; and
b) a switch for activating said shielding mechanism.

8. The system of claim 7 wherein said shielding mechanism comprises:
a) a protective covering positioned upon said distal end of said electrocautery instrument, said protective covering being operatively transitional between a first retracted configuration wherein said tip of said electrocautery element is caused to extend therefrom, and a second operative configuration wherein said shield extends distally beyond said cutting tip of said cutting element; and
b) said switch for activating said shielding mechanism comprises a switch operatively coupled to said control unit and operative to cause said shield mechanism to transition from its first retracted state to second operative state upon receipt of a signal from said control unit.

9. The system of claim 7 wherein said shielding mechanism comprises a solenoid disposed within said housing of said electrocautery device and operatively coupled to said cautery element disposed therein, said solenoid being operative to cause said cutting element to retract within said electrocautery housing.
